(19) United States
(12) Patent Application Publication
(10) Pub. No.: US 2010/0152582 A1
(43) Pub. Date: Jun. 17, 2010

(54) HANDHELD SYSTEM AND METHOD FOR DELIVERING ENERGY TO TISSUE

(75) Inventors: Hira V. Thapliyal, Los Altos, CA (US); David A. Gallup, Alameda, CA (US); James W. Arenson, Woodside, CA (US)

Correspondence Address: VytronUS, Inc. & Townsend and Townsend nd Crew LLP;Joint CN, Two Embarcadero Center, Eighth Floor, San Francisco, CA, 94111, US

(73) Assignee: VytronUS, Inc., Sunnyvale, CA (US)

(21) Appl. No.: 12/483,174

(22) Filed: Jun. 11, 2009

Related U.S. Application Data
(60) Provisional application No. 61/061,548, filed on Jun. 13, 2008.

(51) Int. Cl. A61N 7/02; A61B 18/18; A61B 18/02; A61B 18/04; A61B 8/00

(52) U.S. Cl. 600/439; 601/3; 606/33; 606/14; 606/21; 606/27

(57) ABSTRACT

A system for ablating tissue comprises an ergonomically shaped handpiece having a proximal end and a distal end. An energy source is near the distal end of the handpiece and is adapted to deliver energy to the tissue and create a zone of ablation that blocks abnormal electrical activity in the tissue. The system also includes a barrier near a front face of the energy source. The barrier is adapted to prevent direct contact between blood and the energy source so that the blood does not coagulate on the front face.

### CROSS-REFERENCES TO RELATED APPLICATIONS

This application is a non-provisional of, and claims the benefit of priority under 35 U.S.C. §119(e) of U.S. Provisional Application No. 61/061,548 (Attorney Docket No. 027680-000400US) filed Jun. 13, 2008, the entire contents of which are incorporated herein by reference.

### BACKGROUND OF THE INVENTION

1. Field of the Invention

The present invention relates generally to medical devices, systems and methods, and more specifically to improved devices, systems and methods for creating an ablation zone in tissue. The device may be used to treat atrial fibrillation.

The condition of atrial fibrillation (AF) is characterized by the abnormal (usually very rapid) beating of left atrium of the heart which is out of synch with the normal synchronous movement (“normal sinus rhythm”) of the heart muscle. In normal sinus rhythm, the electrical impulses originate in the sino-atrial node (“SA node”) which resides in the right atrium. The abnormal beating of the atrial heart muscle is known as fibrillation and is caused by electrical impulses originating instead in the pulmonary veins (“PV”) [Haissaguerre, M. et al., Spontaneous Initiation of Atrial Fibrillation by Ectopic Beats Originating in the Pulmonary Veins, New England J Med., Vol. 339:659-666].

There are pharmacological treatments for this condition with varying degrees of success. In addition, there are surgical interventions aimed at removing the aberrant electrical pathways from the PV to the left atrium (“LA”) such as the Cox-Maze III Procedure [J. L. Cox et al., The development of the Maze procedure for the treatment of atrial fibrillation, Seminars in Thoracic & Cardiovascular Surgery, 2000; 12: 2-14; J. L. Cox et al., Electrophysiologic basis, surgical development, and clinical results of the maze procedure for atrial flutter and atrial fibrillation, Advances in Cardiac Surgery, 1995; 6: 1-67; and J. L. Cox et al., Modification of the maze procedure for atrial flutter and atrial fibrillation. II, Surgical technique of the maze III procedure, Journal of Thoracic & Cardiovascular Surgery, 1995; 2110:485-95]. This procedure is shown to be 99% effective [J. L. Cox, N. Ad, T. Palazzo, et al. Current status of the Maze procedure for the treatment of atrial fibrillation, Seminars in Thoracic & Cardiovascular Surgery, 2000; 12: 15-19] but requires special surgical skills and is time consuming.

There has been considerable effort to copy the Cox-Maze procedure for a less invasive percutaneous catheter-based approach. Less invasive treatments have been developed which involve use of some form of energy to ablate (or kill) the tissue surrounding the aberrant focal point where the abnormal signals originate in the PV. The most common methodology is the use of radio-frequency (“RF”) electrical energy to heat the muscle tissue and thereby ablate it. The aberrant electrical impulses are then prevented from traveling from the PV to the atrium (achieving conduction block within the heart tissue) and thus avoiding the fibrillation of the atrial muscle. Other energy sources, such as microwave, laser, and ultrasound have been utilized to achieve the conduction block. In addition, techniques such as cryoablation, administration of ethanol, and the like have also been used.

There has been considerable effort in developing catheter based systems for the treatment of AF using radiofrequency (RF) energy. One such method is described in U.S. Pat. No. 6,064,902 to Haissaguerre et al. In this approach, a catheter is made of distal and proximal electrodes at the tip. The catheter can be bent in a J shape and positioned inside a pulmonary vein. The tissue of the inner wall of the PV is ablated in an attempt to kill the source of the aberrant heart activity. Other RF based catheters are described in U.S. Pat. Nos. 6,814,733 to Schwartz et al., 6,996,908 to Maguire et al., 6,955,173 to Lesh, and 6,949,097 to Stewart et al.

Another source used in ablation is microwave energy. One such device is described by Dr. Mark Levinson [(Endocardial Microwave Ablation: A New Surgical Approach for Atrial Fibrillation; The Heart Surgery Forum, 2006] and Maessen et al. [Beating heart surgical treatment of atrial fibrillation with microwave ablation. Ann Thorac Surg 74: 1160-8, 2002]. This intraoperative device consists of a probe with a malleable antenna which has the ability to ablate the atrial tissue. Other microwave based catheters are described in U.S. Pat. Nos. 4,641,649 to Walinsky; 5,246,438 to Langberg; 5,405,346 to Grundy et al.; and 5,314,466 to Stem et al.

Another catheter based method utilizes the cryogenic technique where the tissue of the atrium is frozen below a temperature of −60 degrees C. This results in killing of the tissue in the vicinity of the PV thereby eliminating the pathway for the aberrant signals causing the AF [A. M. Gillinov, E. H. Blackstone and P. M. McCarthy, Atrial fibrillation: current surgical options and their assessment, Annals of Thoracic Surgery 2002; 74:2210-7]. Cryo-based techniques have been a part of the partial Maze procedures [Sueda T., Nagata H., Orihashi K. et al., Efficacy of a simple left atrial procedure for chronic atrial fibrillation in mitral valve operations, Ann Thorac Surg 1997; 63:1070-1075; and Sueda T., Nagata H., Shikata H. et al.; Simple left atrial procedure for chronic atrial fibrillation associated with mitral valve disease, Ann Thorac Surg 1996; 62: 1796-1800]. More recently, Dr. Cox and his group [Nathan H., Eliakim M., The junction between the left atrium and the pulmonary veins, An anatomic study of human hearts, Circulation 1966; 34:412-422, and Cox J. L., Schuessler R. B., Boineau J. P., The development of the Maze procedure for the treatment of atrial fibrillation, Semin Thorac Cardiovasc Surg 2000; 12:2-14] have used cryoprobes (cryo-Maze) to duplicate the essentials of the Cox-Maze III procedure. Other cryo-based devices are described in U.S. Pat. Nos. 6,929,639 and 6,666,858 to Lafintaine and 6,161,543 to Cox et al.

More recent approaches for the AF treatment involve the use of ultrasound energy. The target tissue of the region surrounding the pulmonary vein is heated with ultrasound energy emitted by one or more ultrasound transducers. One such approach is described by Lesh et al. in U.S. Pat. No. 6,502,576. Here the catheter distal tip portion is equipped with a balloon which contains an ultrasound element. The balloon serves as an anchoring means to secure the tip of the catheter in the pulmonary vein. The balloon portion of the catheter is positioned in the selected pulmonary vein and the balloon is inflated with a fluid which is transparent to ultrasound energy. The transducer emits the ultrasound energy which travels to the target tissue in or near the pulmonary vein and ablates it. The intended therapy is to destroy the electrical conduction path around a pulmonary vein and thereby restore the normal sinus rhythm. The therapy involves the creation of a multiplicity of lesions around individual pulmonary veins as required. The inventors describe various configurations for the energy emitter and the anchoring mechanisms.

Yet another catheter device using ultrasound energy is described by Gentry et al. [Integrated Catheter for 3-D Intracardiac Echocardiography and Ultrasound Ablation, IEEE Transactions on Ultrasonics, Ferroelectrics, and Frequency Control, Vol. 51, No. 7, pp 799-807]. Here the catheter tip is made of an array of ultrasound elements in a grid pattern for the purpose of creating a three dimensional image of the target tissue. An ablating ultrasound transducer is provided which is in the shape of a ring which encircles the imaging grid. The ablating transducer emits a ring of ultrasound energy at 10 MHz frequency. In a separate publication [Medical Device Link, Medical Device and Diagnostic Industry, February 2006], in the description of the device, the authors assert that the pulmonary veins can be imaged.

While these devices and methods are promising, improved devices and methods for creating a heated zone of tissue, such as an ablation zone are needed. Furthermore, it would also be desirable if such devices could create single or multiple ablation zones to block abnormal electrical activity in the heart in order to lessen or prevent atrial fibrillation. It would also be desirable if such devices could be used in the presence of blood or other body tissues without coagulating or clogging up the ultrasound transducer. Such devices and methods should be easy to use, cost effective and simple to manufacture.

2. Description of Background Art

Other devices based on ultrasound energy to create circumferential lesions are described in U.S. Pat. Nos. 6,997,925; 6,966,908; 6,964,660; 6,954,977; 6,953,460; 6,652,515; 6,547,788; and 6,514,249 to Maguire et al.; 6,955,173; 6,052,576; 6,305,378; 6,164,283; and 6,012,457 to Lesh; 6,872,205; 6,416,511; 6,254,599; 6,245,064; and 6,024,740; to Lesh et al.; 6,383,151; 6,117,101; and WO 99/02096 to Diederich et al.; 6,635,054 to Fjield et al.; 6,780,183 to Jimenez et al.; 6,605,084 to Acker et al.; 5,295,484 to Marcus et al.; and WO 2005/117734 to Wong et al.

In all above approaches, the inventions involve the ablation of tissue inside a pulmonary vein or at the location of the ostium. The anchoring mechanisms engage the inside lumen of the target pulmonary vein. In all these approaches, the anchor is placed inside one vein, and the ablation is done one vein at a time.

### BRIEF SUMMARY OF THE INVENTION

The present invention relates generally to medical devices, systems and methods, and more specifically to devices, systems and methods for ablating tissue.

In a first aspect of the present invention, a system for ablating tissue in a patient comprises a handpiece having a proximal end and a distal end. The handpiece is ergonomically shaped to fit in an operator's hand. An energy source is disposed near the distal end of the handpiece and is adapted to deliver energy to the tissue. This creates a zone of ablation that blocks abnormal electrical activity in the tissue. A barrier is near a front face of the energy source and the barrier prevents direct contact between blood and the energy source so that the blood does not coagulate on the front face.

The handpiece may comprise a flexible shaft that is bendable into a desired configuration. The system may comprise a bending mechanism such as a wire, that is operably coupled with the shaft and adapted to bend the shaft. The handpiece may also have a rigid shaft. The handpiece may comprise an elongate shaft having one or more lumens extending therethrough. A portion of the handpiece near the distal end may be transparent to the energy emitted from the energy source. Also, a portion of the handpiece near the distal end may comprise a plurality of apertures adapted to allow fluid flow therethrough. The plurality of apertures may comprise a series of castellated slots. A distal portion of the handpiece may also define a fixed path along which the energy source may be moved. The fixed path may comprise an arcuate shape such as a loop or the path may comprise a linear region.

The energy may be delivered at an angle relative to the tissue, the angle being between 65 degrees and 115 degrees. The energy source may comprise an ultrasound transducer. The energy source may deliver one of radiofrequency energy, microwave energy, photonic energy, thermal energy, and cryogenic energy. The energy source may comprise a plurality of energy sources. The energy source may comprise a backing material coupled therewith and that provides a heat sink for the energy source. The backing may comprise an outer wall having a plurality of longitudinally oriented grooves adapted to allow cooling fluid to flow therethrough. Also, an air pocket may be disposed between the backing material and the energy source. The backing material may also be adapted to reflect energy from the energy source distally toward the distal end of the handpiece. The energy source may be movable proximally and distally relative to the distal end of the handpiece. The energy source may also be rotatably moveable in the handpiece.

The barrier may comprise a fluid flowing past the energy source. The zone of ablation may block abnormal electrical activity thereby reducing or eliminating atrial fibrillation in the patient. The tissue may comprise tissue in an atrium of the patient's heart, a pulmonary vein or tissue adjacent the a pulmonary vein. A gap may separate the energy source from a surface of the tissue, the gap may range from 1 mm to 15 mm. The system may also include a cooling mechanism for cooling the energy source. The cooling mechanism may comprise a fluid flowing past the energy source. The cooling mechanism may also comprise a fluid flowing into contact with the tissue thereby altering the shape or depth of the zone of ablation. The system may include a sensor that is adapted to detect the gap between the energy source and a surface of the tissue. The sensor may also be adapted to determine the thickness of the tissue. The energy source may comprise an ultrasound transducer and the sensor also may comprise the same ultrasound transducer of the energy source.

In another aspect of the present invention, an ultrasound system for ablating tissue in a patient comprises a handpiece having a proximal end, a distal end, and a fixed path near the distal end. The handpiece is ergonomically shaped to fit in an operator's hand. An ultrasound transducer is near the distal end of the handpiece, and is adapted to deliver energy to the tissue and create a zone of ablation that blocks abnormal electrical activity in the tissue, thereby reducing or eliminating atrial fibrillation in the patient. The transducer is movable along the fixed path and the system also has a barrier near a front face of the transducer. The barrier is adapted to prevent direct contact between blood and the transducer so that the blood does not coagulate on the front face.

In still another aspect of the present invention, a method of ablating tissue in a patient comprises providing an ultrasound treatment device having a handpiece and positioning a distal portion of the handpiece adjacent the tissue. Ultrasound energy is delivered from an ultrasound transducer near the distal end of the handpiece to the tissue and a zone of ablation is created in the tissue. The ablation zone blocks abnormal electrical activity in the tissue thereby reducing or eliminating atrial fibrillation in the patient. A barrier is maintained near a front face of the transducer thereby preventing direct contact between blood and the transducer so as to prevent coagulation of the blood on the front face.

The step of positioning may comprise positioning the distal portion of the handpiece adjacent the patient's heart and the tissue may comprise tissue in an atrium of the patient's heart, a pulmonary vein or tissue adjacent a pulmonary vein. The step of positioning may comprise adjusting an angle between the handpiece and the tissue, thereby adjusting direction of the energy from the transducer to the tissue.

Creating the zone of ablation may comprise moving the transducer proximally and distally relative to a distal end of the handpiece or rotating the transducer in the handpiece. The handpiece may comprise a fixed path near a distal end thereof, and the step of creating the zone of ablation may comprise moving the transducer along the fixed path. The fixed path may comprise a loop.

The method may further comprise moving the handpiece along a surface of the tissue, thereby increasing the zone of ablation. The method may also include bending the handpiece into a desired configuration. The transducer may be cooled with a fluid and the fluid may flow past the transducer at a flow rate high enough to prevent blood from contacting the transducer. The tissue may also be cooled with a fluid in order to alter the shape or depth of the zone of ablation. The method may further comprise maintaining a gap between the transducer and the tissue. The gap may range from 1 mm to 15 mm. The method may further comprise sensing distance between the transducer and the tissue with a sensor disposed near a distal end of the handpiece and the distance between the transducer and the tissue may be adjusted as required. The sensor may also be used to sense tissue characteristics such as tissue depth.

These and other embodiments are described in further detail in the following description related to the appended drawing figures.

### BRIEF DESCRIPTION OF THE DRAWINGS

FIG. 1 is a drawing of the system of the preferred embodiments of the invention; and

FIGS. 2-4 are drawings of a first, second, and third variation, respectively, of the distal tip assembly of the system of the preferred embodiments of the invention.

### DETAILED DESCRIPTION OF THE INVENTION

The following description of preferred embodiments of the invention is not intended to limit the invention to these embodiments, but rather to enable any person skilled in the art to make and use this invention.

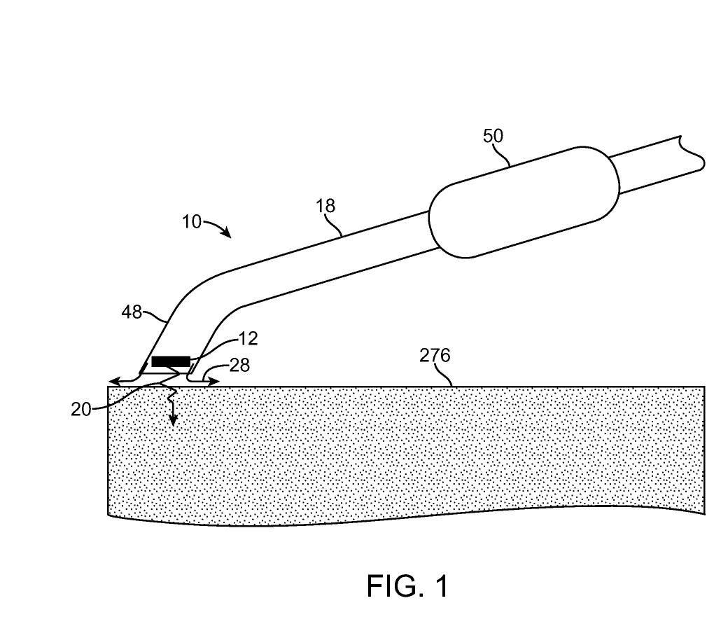

As shown in FIG. 1, the handheld system 10 of the preferred embodiments includes an elongate member 18 having a distal tip assembly 48 and a handle 50. The distal tip assembly 48, which preferably includes an energy source 12, functions to direct energy to a tissue 276. The handheld system 10 is preferably designed for delivering energy to tissue, more specifically, for delivering ablation energy to tissue, such as heart tissue, including an atrium of the heart, a pulmonary vein or tissue adjacent the pulmonary vein, to create an ablated tissue zone which results in a conduction block-isolation and/or block of conduction pathways of abnormal electrical activity, which typically originate from the pulmonary veins in the left atrium for treatment of atrial fibrillation in a patient. The handheld system 10, however, may be alternatively used with any suitable tissue in any suitable environment and for any suitable reason.

The Elongate Member. As shown in FIG. 1, the elongate member 18 of the preferred embodiments is preferably a shaft having a distal tip assembly 48 and a handle 50. The elongate member 18 preferably couples the handle 50 to the distal tip assembly 48, such that the distal tip assembly 48 (and/or energy source 12) can be moved along a surface of tissue 276. The shaft is preferably a flexible shaft, such that it can be bent and positioned into a desired configuration. The shaft preferably remains in the desired configuration until it is re-bent or re-positioned into an alternative desired configuration. The elongate member 18 may further include a bending mechanism that functions to bend or position the elongate member 18 at various locations (such as bending a distal portion of the elongate member 18 towards the tissue 276, as shown in FIG. 1). The bending mechanism preferably includes lengths of wires, ribbons, cables, lines, fibers, filament or any other tensional member. Alternatively, the elongate member 18 may be a fixed or rigid shaft or any other suitable shaft, such as a gooseneck type shaft that includes a plurality of sections, aligned axially, that move with respect to one another to bend and position the shaft. The shaft is preferably a multilumen tube, but may alternatively be a catheter, a cannula, a tube or any other suitable elongate structure having one or more lumens. The elongate member 18 of the preferred embodiments functions to accommodate pull wires, fluids, gases, energy delivery structures, electrical connections, and/or any other suitable device or element.

The Distal Tip Assembly. As shown in FIG. 1, the elongate member 18 of the preferred embodiments preferably includes a distal tip assembly 48 at a distal portion of the elongate member 18. The distal tip assembly 48 functions to direct energy to a tissue 276 and preferably houses an energy source 12 that functions to provide a source of ablation energy and emits an energy beam 20. The distal tip assembly 48, and the energy source 12 within it, are preferably moved and positioned within a patient, preferably within the left atrium of the heart of the patient, such that the distal tip assembly 48 directs the emitted energy beam 20 from the energy source 12 to a tissue 276 and such that energy beam 20 contacts the target tissue 276 at an appropriate angle. The emitted energy beam 20 preferably contacts the target tissue at an angle between 20 and 160 degrees to the tissue, more preferably contacts the target tissue at an angle between 45 and 135 degrees to the tissue, and most preferably contacts the target tissue at an angle of 65 and 115 degrees to the tissue.

The energy source 12 is preferably an ultrasound transducer that emits an ultrasound beam, but may alternatively be any suitable energy source that functions to provide a source of ablation energy. Some suitable sources of ablation energy include radio frequency (RF) energy, microwaves, photonic energy, and thermal energy. The therapy could alternatively be achieved using cooled fluids (e.g., cryogenic fluid). The distal tip assembly 48 preferably includes a single energy source 12, but may alternatively include any suitable number of energy sources 12. The ultrasound transducer is preferably made of a piezoelectric material such as PZT (lead zirconate titanate) or PVDF (polyvinylidine difluoride), or any other suitable ultrasound beam emitting material. The transducer may further include coating layers such as a thin layer of a metal. Some suitable transducer coating metals may include gold, stainless steel, nickel-cadmium, silver, and a metal alloy.

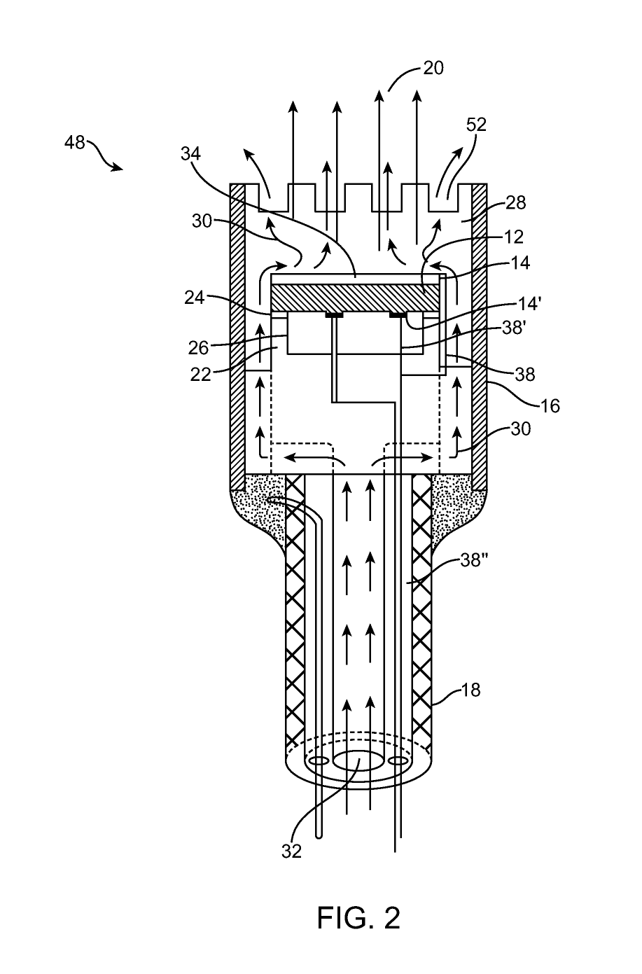

As shown in FIG. 2, the distal tip assembly 48 of the preferred embodiments also includes a backing 22, coupled to the energy source 12. The energy source 12 is preferably bonded to the end of a backing 22 by means of an adhesive ring 24. The backing 22 is preferably made of a metal or a plastic, such that it provides a heat sink for the energy source 12. The attachment of the energy source 12 to the backing 22 is such that there is a pocket 26 between the back surface of the energy source 12 and the backing 22. The pocket is preferably one of several variations. In a first version, the backing 22 couples to the energy source at multiple points. For example, the backing preferably includes three posts that preferably couple to the outer portion such that the majority of the energy source 12 is not touching a portion of the backing. In this variation, a fluid or gel preferably flows past the energy source 12, bathing preferably both the front and back surfaces of the energy source 12. In a second variation, the pocket is an air pocket 26 between the back surface of the energy source 12 and the backing 22. The air pocket 26 functions such that when the energy source 12 is energized by the application of electrical energy, the emitted energy beam 20 is reflected by the air pocket 26 and directed outwards from the energy source 12. The backing 22 preferably defines an air pocket of a cylindrical shape, and more preferably defines an air pocket 26 that has an annular shape. The backing defines an annular air pocket by further including a center post such that the backing has a substantially tripod shape when viewed in cross section, wherein the backing is coupled to the energy source 12 towards both the outer portion of the energy source and towards the center portion of the energy source. The air pocket 26 may be replaced by any other suitable material such that a substantial portion of the energy beam 20 is directed outwards from the energy source 12.

While the energy source 12 is emitting an energy beam 20, the energy source may become heated. The energy source 12 is preferably maintained within an optimal operating temperature range by cooling the energy source 12. Cooling of the energy source 12 is preferably accomplished by contacting the energy source 12 with a fluid, for example, saline or any other physiologically compatible fluid or gel, preferably having a lower temperature relative to the temperature of the energy source 12. The temperature of the fluid or gel is preferably between −5 and 5 degrees Celsius and more preferably substantially equal to zero degrees Celsius. The fluid may alternatively be any suitable temperature to sufficiently cool the energy source 12 and/or to alter the physical characteristics, such as shape and depth, of the zone of ablated tissue created by the interaction between tissue and the energy beam 20 emitted from the energy source 12. The backing 22 preferably has a series of grooves disposed longitudinally along the outside wall that function to provide for the flow of a cooling fluid 28 substantially along the outer surface of backing 22 and past the face of the energy source 12. The series of grooves may alternatively be disposed along the backing in any other suitable configuration, such as helical. The resulting fluid flow lines are depicted as 30 in FIG. 2. The flow of the cooling fluid is achieved through a lumen 32.

As shown in FIG. 2, the distal tip assembly 48 preferably includes a housing 16 coupled to the energy source 12. The housing is preferably an open, tubular housing 16, but may alternatively be a closed end housing that encloses the energy source 12. At least a portion of the closed end housing is made of a material that is transparent to the energy beam 20. The material is preferably transparent to ultrasound energy, such as a poly 4-methyl, 1-pentene (PMP) material or any other suitable material. As shown in FIG. 2, the open tubular housing preferably has a “castle head” configuration having slots 52. The slots 52 function to provide exit ports for the flowing fluid 28. When the front tip of the distal tip assembly 48 is in contact with or adjacent to the tissue 276 or other structures during the use of the handheld system 10, the slots 52 function to maintain the flow of the cooling fluid 28 past the energy source 12 and along the surface of the tissue 276. The fluid flow lines 30 flow along the grooves in the backing 22, bathe the energy source 12, form a fluid column and exit through the slots 52 at the castle head housing 16. In the closed end housing, the housing includes apertures such as small holes towards the distal end of the housing 16. These holes provide for the exit path for the flowing fluid. The apertures are preferably a grating, screen, holes, drip holes, weeping structure or any other suitable apertures. Alternatively, the closed end housing may not have apertures to allow the exit of the fluid but rather contains the fluid or gel within the housing and recycles the fluid past the energy source 12.

The housing 16 of the distal tip assembly 48, further functions to provide a barrier between the face of the energy source 12 and the blood residing in the patient, such as in the atrium of the heart. If the fluid flow is not incorporated, and the transducer face is directly in contact with blood, the blood will coagulate on the surface of the energy source 12. Additionally, there is a possibility of forming a blood clot at the interface of the energy source 12 and the surrounding blood. The flow of the cooling fluid 28 keeps the blood from contacting the energy source 12, thus avoiding the formation of blood clots. The flow rate is preferably 1 ml per minute, but may alternatively be any other suitable flow rate to maintain the fluid column, keep the separation between the blood and the face of the energy source 12, cool the energy source 12, and/or cool the tissue 276. Additional details about housing 16 and the components therein are disclosed in greater detail in U.S. patent application Ser. No. 12/480,256 (Attorney Docket No. 027680-000310US), filed Jun. 8, 2009, the entire contents of which are incorporated herein by reference.

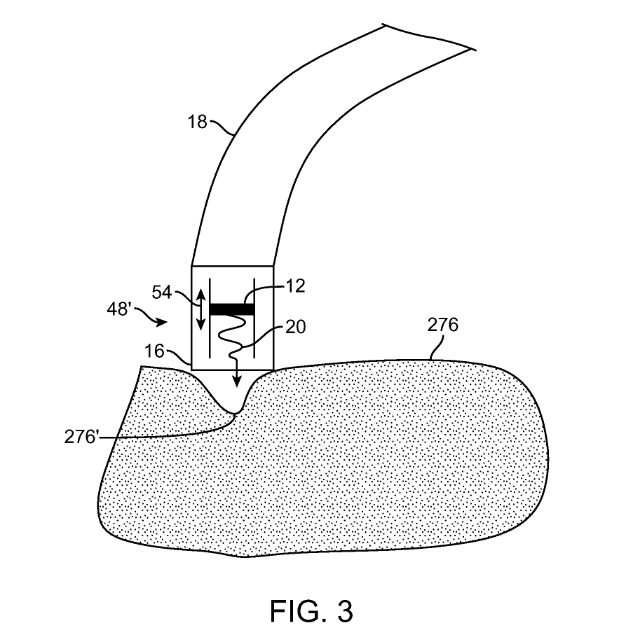

The distal tip assembly 48 is preferably one of several variations. In a first variation, as shown in FIG. 2, the energy source 12 is fixed within the distal tip assembly 48, a distance from the distal tip of the housing 16. In a second variation, as shown in FIG. 3, the energy source 12 is moveable within the distal tip assembly 48′ with respect to the distal tip of the housing 16. The energy source 12 is preferably moved closer to and further from the distal tip housing 16, as shown by arrows 54. The energy source 12 may additionally be rotated such that the energy beam 20 exits at an angle with respect to the longitudinal axis of the housing 16. The energy source 12 is preferably moved with respect to the housing 16 such that the beam emitted 20 from the energy source 12 preferably contacts the tissue at an appropriate angle and such that the energy source is an appropriate distance from the surface of the tissue, i.e. the gap distance. The emitted energy beam 20 preferably contacts the target tissue at an angle between 20 and 160 degrees to the tissue, more preferably contacts the target tissue at an angle between 45 and 135 degrees to the tissue, and most preferably contacts the target tissue at an angle of 65 and 115 degrees to the tissue. The surface of tissue is not always flat, it occasionally has ridges and/or creases, as shown in FIG. 3. When the surface of the tissue 276 is not substantially flat, as the operator and/or motor drive unit (not shown) is guiding the system 10 over the surface of the tissue, the distal tip of the system may not fit into all contours of the tissue, such as crease 276′. In this situation, the energy source 12 is preferably moved closer to the distal tip of the distal tip assembly 48, such that the energy source 12 maintains an appropriate gap distance from the surface of the tissue. The gap distance is preferably between 1 mm and 20 mm, and more preferably between 1 mm and 15 mm.

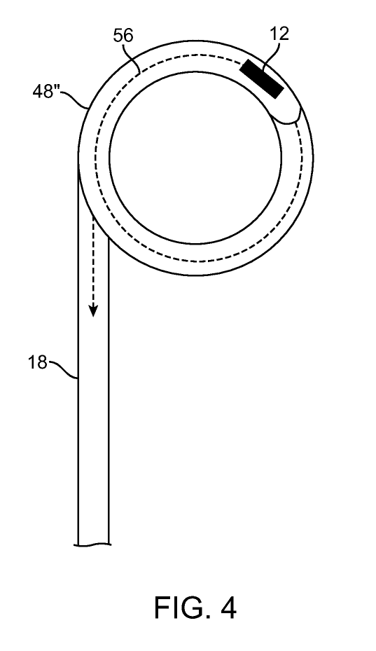

In a third variation, as shown in FIG. 4, the distal tip assembly 48″ defines a fixed path 56 along which the energy source 12 is positioned. The fixed path 56 is preferably circular or elliptical such that it encircles at least one pulmonary vein, but may alternatively be any other suitable geometry and may enclose any suitable number of pulmonary veins. The fixed path 56 may alternatively be linear or curved. The fixed path may also be used to treat other tissue, such as atrial tissue, tissue adjacent a pulmonary vein or other tissues. The distal tip assembly 48″ is preferably movable and positionable such that the fixed path 56 takes on any suitable geometry. In this variation, the energy source 12 is preferably pushed or pulled along the fixed path 56 within the distal tip assembly. The energy source 12 is preferably energized such that it emits an energy beam as it is moved along the fixed path 56 through the distal tip assembly. Alternatively, the energy source may be energized in a single location along the fixed path 56 within the distal tip assembly 48″. While energized in a single location, the distal tip assembly 48″ may then be moved along an ablation path. The distal tip assembly 48″ preferably includes apertures along its length, to maintain fluid flow as described above.

The Handle. As shown in FIG. 1, the elongate member 18 of the preferred embodiments preferably includes a handle 50 at a proximal portion of the elongate member 18. The handle 50 functions to provide a portion where an operator and/or motor drive unit couples to the system 10. The handle 50 is preferably held and moved by an operator holding the handle 50, but alternatively, the handle 50 is coupled to a motor drive unit and the movements are preferably computer controlled movements. The handle 50 may alternatively be coupled and moved in any other suitable fashion. While coupled to the handle 50 of the handheld system 10, an operator and/or motor drive unit moves the distal tip assembly 48, and/or the energy source 12, along a surface of tissue 276. The distal tip assembly 48, and the energy source 12 within it, are preferably moved and positioned within a patient, preferably within the left atrium of the heart of the patient, such that the distal tip assembly 48 directs the emitted energy beam 20 from the energy source 12 to a tissue 276 and such that energy beam 20 contacts the target tissue 276 at an appropriate angle. The operator and/or motor drive unit preferably moves the handheld system 10 along an ablation path, similarly to moving a pen across a writing surface, and energizes the energy source 12 to emit energy beam 20 such that the energy source 12 provides a partial or complete zone of ablation along the ablation path. The zone of ablation along the ablation path preferably has any suitable geometry to provide therapy, such as providing a conduction block for treatment of atrial fibrillation in a patient. The zone of ablation along the ablation path may alternatively provide any other suitable therapy for a patient.

The handle 50 is preferably one of several variations. In a first variation, as shown in FIG. 1, the handle 50 is a raised portion on the elongate member 18, alternatively, the handle 50 may simply be a proximal portion of the elongate member 18 held by the operator. The handle 50 may further include finger recesses, or any other suitable ergonomic grip geometry. The handle is preferably made of a material with a high coefficient of friction, such as rubber, foam, or plastic, such that the handle 50 does not slip from the operator's hand. The handle 50 may further include controls such as dials, buttons, and an output display such that the operator may control the energy source 12, the position of the energy source 12, the sensor (described below), the fluid flow, the bending mechanism, and/or any other suitable element of device of the hand held system 10. The handle 50 may be removably coupled to a motor drive unit or may alternatively be integrated directly into the motor drive unit.

The Sensor. The distal tip assembly 48 of the preferred embodiments also includes a sensor that functions to detect the gap (namely, the distance of the tissue surface from the energy source 12), the thickness of the tissue 276 targeted for ablation, the characteristics of the ablated tissue, and any other suitable parameter or characteristic. The sensor is preferably an ultrasound transducer, but may alternatively be any suitable sensor to detect the gap, the thickness of the tissue targeted for ablation, the characteristics of the ablated tissue, and any other suitable parameter or characteristic. The ultrasound transducer preferably utilizes a pulse of ultrasound of short duration, which is generally not sufficient for heating of the tissue. This is a simple ultrasound imaging technique, referred to in the art as A Mode, or Amplitude Mode imaging. The sensor is preferably the same transducer as the transducer of the energy source, operating in a different mode (such as A-mode), or may alternatively be a separate ultrasound transducer. By detecting information on the gap, the thickness of the tissue targeted for ablation, and the characteristics of the ablated tissue, the sensor preferably functions to guide the therapy provided by the ablation of the tissue and guide the operator and/or motor drive unit as to where to position the handheld system, at what position to have the energy source with respect to the distal tip assembly in order to maintain a proper gap distance, and at what settings at which to use the energy source 12 and any other suitable elements.

Although omitted for conciseness, the preferred embodiments include every combination and permutation of the various elongate members 18, distal tip assemblies 48, energy sources 12, and handles 50.

As a person skilled in the art will recognize from the previous detailed description and from the figures and claim, modifications and changes can be made to the preferred embodiments of the invention without departing from the scope of this invention defined in the following claims.

What is claimed is:

1. A system for ablating tissue in a patient, said system comprising:
a handpiece having a proximal end and a distal end, the handpiece ergonomically shaped to fit in an operator's hand;
an energy source near the distal end of the handpiece, the energy source adapted to deliver energy to the tissue and create a zone of ablation that blocks abnormal electrical activity in the tissue; and
a barrier near a front face of the energy source, the barrier adapted to prevent direct contact between blood and the energy source so that the blood does not coagulate on the front face.

2. The system of claim 1, wherein the handpiece comprises a flexible shaft, the shaft being bendable into a desired configuration.

3. The system of claim 2, further comprising a bending mechanism operably coupled with the shaft and adapted to bend the shaft.

4. The system of claim 3, wherein the bending mechanism comprises a wire.

5. The system of claim 1, wherein the handpiece comprises a rigid shaft.

6. The system of claim 1, wherein the handpiece comprises an elongate shaft having one or more lumens extending therethrough.

7. The system of claim 1, wherein a portion of the handpiece near the distal end is transparent to the energy emitted from the energy source.

8. The system of claim 1, wherein a portion of the handpiece near the distal end comprises a plurality of apertures adapted to allow fluid flow therethrough.

9. The system of claim 8, wherein the plurality of apertures comprise a series of castellated slots.

10. The system of claim 1, wherein a distal portion of the handpiece defines a fixed path along which the energy source is movable.

11. The system of claim 10, wherein the fixed path comprises an arcuate shape.

12. The system of claim 1, wherein the energy is delivered at an angle relative to the tissue, the angle being between 65 degrees and 115 degrees.

13. The system of claim 1, wherein the energy source comprises an ultrasound transducer.

14. The system of claim 1, wherein the energy source delivers one of radiofrequency energy, microwave energy, photonic energy, thermal energy, and cryogenic energy.

15. The system of claim 1, wherein the energy source comprises a plurality of energy sources.

16. The system of claim 1, wherein the energy source comprises a backing material coupled therewith, the backing material providing a heat sink for the energy source.

17. The system of claim 16, wherein the backing comprises an outer wall having a plurality of longitudinally oriented grooves adapted to allow cooling fluid to flow therethrough.

18. The system of claim 16, wherein an air pocket is disposed between the backing material and the energy source.

19. The system of claim 1, wherein the energy source comprises a backing material, the backing material adapted to reflect energy from the energy source distally toward the distal end of the handpiece.

20. The system of claim 1, wherein the energy source is movable proximally and distally relative to the distal end of the handpiece.

21. The system of claim 1, wherein the energy source is rotatably moveable in the handpiece.

22. The system of claim 1, wherein the barrier comprises a fluid flowing past the energy source.

23. The system of claim 1, wherein the zone of ablation blocks abnormal electrical activity thereby reducing or eliminating atrial fibrillation in the patient.

24. The system of claim 1, wherein the tissue comprises a pulmonary vein.

25. The system of claim 1, wherein the tissue comprises tissue adjacent a pulmonary vein.

26. The system of claim 1, wherein the tissue comprises atrial tissue.

27. The system of claim 1, wherein a gap separates the energy source from a surface of the tissue.

28. The system of claim 27, wherein the gap ranges from 1 mm to 15 mm.

29. The system of claim 1, further comprising a cooling mechanism for cooling the energy source.

30. The system of claim 29, wherein the cooling mechanism comprises a fluid flowing past the energy source.

31. The system of claim 29, wherein the cooling mechanism comprises a fluid flowing into contact with the tissue thereby altering the shape or depth of the zone of ablation.

32. The system of claim 1, further comprising a sensor adapted to detect a gap between the energy source and a surface of the tissue.

33. The system of claim 32, wherein the sensor is adapted to determine thickness of the tissue.

34. The system of claim 32, wherein the energy source comprises an ultrasound transducer and the sensor also comprises the same ultrasound transducer of the energy source.

35. An ultrasound system for ablating tissue in a patient, said system comprising:
a handpiece having a proximal end, a distal end, and a fixed path near the distal end, the handpiece ergonomically shaped to fit in an operator's hand;
an ultrasound transducer near the distal end of the handpiece, the transducer adapted to deliver energy to the tissue and create a zone of ablation that blocks abnormal electrical activity in the tissue, thereby reducing or eliminating atrial fibrillation in the patient,
wherein the transducer is movable along the fixed path; and
a barrier near a front face of the transducer, the barrier adapted to prevent direct contact between blood and the transducer so that the blood does not coagulate on the front face.

36. The system of claim 35, wherein the fixed path comprises a loop.

37. The system of claim 35, wherein a portion of the handpiece near the distal end comprises a plurality of apertures adapted to allow fluid flow therethrough.

38. The system of claim 35, wherein the tissue comprises a pulmonary vein.

39. The system of claim 35, wherein the tissue comprises tissue adjacent a pulmonary vein.

40. The system of claim 35, wherein the tissue comprises atrial tissue.

41. The system of claim 35, wherein a gap separates the ultrasound transducer from a surface of the tissue.

42. The system of claim 41, wherein the gap ranges from 1 mm to 15 mm.

43. The system of claim 35, further comprising a cooling mechanism for cooling the transducer.

44. The system of claim 35, further comprising a sensor adapted to detect a gap between the energy source and a surface of the tissue.

45. The system of claim 44, wherein the sensor is adapted to determine thickness of the tissue.

46. A method of ablating tissue in a patient, said method comprising:
providing an ultrasound treatment device having a handpiece;
positioning a distal portion of the handpiece adjacent the tissue;
delivering ultrasound energy from an ultrasound transducer near the distal end of the handpiece to the tissue;
creating a zone of ablation in the tissue that blocks abnormal electrical activity in the tissue thereby reducing or eliminating atrial fibrillation in the patient; and
maintaining a barrier near a front face of the transducer thereby preventing direct contact between blood and the transducer so as to prevent coagulation of the blood on the front face.

47. The method of claim 46, wherein the step of positioning comprises positioning the distal portion of the handpiece adjacent the patient's heart.

48. The method of claim 46, wherein the tissue comprises a pulmonary vein.

49. The method of claim 46, wherein the tissue comprises tissue adjacent a pulmonary vein.

50. The method of claim 46, wherein the tissue comprises atrial tissue.

51. The method of claim 46, wherein the step of positioning comprises adjusting an angle between the handpiece and the tissue, thereby adjusting direction of the energy from the transducer to the tissue.

52. The method of claim 46, wherein the step of creating the zone of ablation comprises moving the transducer proximally and distally relative to a distal end of the handpiece.

53. The method of claim 46, wherein the step of creating the zone of ablation comprises rotating the transducer within the handpiece.

54. The method of claim 46, wherein the handpiece comprises a fixed path near a distal end thereof, and the step of creating the zone of ablation comprises moving the transducer along the fixed path.

55. The method of claim 54, wherein the fixed path comprises a loop.

56. The method of claim 46, further comprising moving the handpiece along a surface of the tissue, thereby increasing the zone of ablation.

57. The method of claim 46, further comprising bending the handpiece into a desired configuration.

58. The method of claim 46, further comprising cooling the transducer with a fluid.

59. The method of claim 58, wherein the fluid flows past the transducer at a flow rate high enough to prevent blood from contacting the transducer.

60. The method of claim 46, further comprising cooling the tissue with a fluid thereby altering the shape or depth of the zone of ablation.

61. The method of claim 46, further comprising maintaining a gap between the transducer and the tissue.

62. The method of claim 61, wherein the gap ranges from 1 mm to 15 mm.

63. The method of claim 46, further comprising sensing distance between the transducer and the tissue with a sensor disposed near a distal end of the handpiece.

64. The method of claim 63, further comprising adjusting the distance between the transducer and the tissue.

65. The method of claim 46, further comprising sensing characteristics of the tissue with a sensor disposed near a distal end of the handpiece.
